SEQUENCE LISTING (17 sequences)

SEQ ID NO: 1 - DNA, 34 bp, Artificial Sequence
  oligonucleotide (MS1, MS2, MS3)
ctttcctacacctacgtctccaactaacttacgg

SEQ ID NO: 2 - DNA, 36 bp, Artificial Sequence
  oligonucleotide (MS1)
ccacatacatcatattccctcattcaataccctacg

SEQ ID NO: 3 - DNA, 50 bp, Artificial Sequence
  oligonucleotide (MS1, MS2, MS3)
tggagacgtagggtattgaatgagggccgtaagttagttggagacgtagg

SEQ ID NO: 4 - DNA, 35 bp, Artificial Sequence
  oligonucleotide (MS2)
ccacatacatcatattccctcattcaataccctac

SEQ ID NO: 5 - DNA, 34 bp, Artificial Sequence
  oligonucleotide (MS3)
ccacatacatcatattccctcattcaatacccta

SEQ ID NO: 6 - DNA, 22 bp, Artificial Sequence
  oligonucleotide (CS)
cattcaataccctacgtctcca

SEQ ID NO: 7 - DNA, 17 bp, Artificial Sequence
  oligonucleotide (CS1)
cattcaataccctacgt

SEQ ID NO: 8 - DNA, 5 bp, Artificial Sequence
  oligonucleotide (CS2)
ctcca

SEQ ID NO: 9 - DNA, 12 bp, Artificial Sequence
  oligonucleotide (xCS1)
cattcaataccc

SEQ ID NO: 10 - DNA, 10 bp, Artificial Sequence
  oligonucleotide (xCS2)
tacgtctcca

SEQ ID NO: 11 - DNA, 44 bp, Artificial Sequence
  oligonucleotide (DS)
cctacgtctccaactaacttacggccctcattcaataccctacg

SEQ ID NO: 12 - DNA, 34 bp, Artificial Sequence; identical to SEQ ID NO: 5
  olionucleotide (WS)

SEQ ID NO: 13 - DNA, 50 bp, Artificial Sequence; identical to SEQ ID NO: 3
  oligonucleotide (WS)

SEQ ID NO: 14 - DNA, 20 bp, Artificial Sequence
  oligonuleotide (TS1Dye)
ccacatacatcatattccct

SEQ ID NO: 15 - DNA, 30 bp, Artificial Sequence
  oligonucleotide (TS1Dye)
gtattgaatgagggaatatgatgtatgtgg

SEQ ID NO: 16 - DNA, 16 bp, Artificial Sequence
  oligonucleotide (TS2Dye)
ctttcctacacctacg

SEQ ID NO: 17 - DNA, 22 bp, Artificial Sequence
  oligonucleotide (TS2Dye)
tggagacgtaggtgtaggaaag

(19) United States
(12) Patent Application Publication
(10) Pub. No.: US 2018/0179576 A1
(43) Pub. Date: Jun. 28, 2018

(54) HOMOGENEOUS ENTROPY-DRIVEN BIOMOLECULAR ASSAY (HEBA)

(71) Applicant: THE REGENTS OF THE UNIVERSITY OF CALIFORNIA, Oakland, CA (US)

(72) Inventors: Dino DI CARLO, Los Angeles, CA (US); Donghyuk Kim, Los Angeles, CA (US)

(73) Assignee: THE REGENTS OF THE UNIVERSITY OF CALIFORNIA, Oakland, CA (US)

(21) Appl. No.: 15/554,397

(22) PCT Filed: Mar. 2, 2016
(86) PCT No.: PCT/US2016/020411

§ 371 Date: Aug. 29, 2017

Related U.S. Application Data
(60) Provisional application No. 62/127,050, filed on Mar. 2, 2015.

(51) Int. Cl. C12Q 1/682; G01N 21/64

(52) U.S. Cl. CPC C12Q 1/682; G01N 2021/6439; G01N 2021/6432; G01N 21/6428

(57) ABSTRACT

A method for detecting an analyte in a sample includes the steps of binding a first catalytic precursor to the analyte at a first epitope and binding a second catalytic precursor to the analyte at a second, different epitope to generate a catalytic complex. The catalytic complex is reacted with a multiplex molecular substrate to generate a first target molecule and an intermediate substrate containing the bound catalytic complex. The intermediate substrate is reacted with a dummy reactant to generate a second target molecule, wherein the reaction further generates a waste molecule containing the dummy reactant and a free catalytic complex. An optical signal that is generated by one or more dye(s) specific to the first target molecule and/or the second target molecule is detected to detect the presence of the analyte in the sample. The overall reaction has substantially net zero enthalpy and a positive entropy change.

### RELATED APPLICATION

This Application claims priority to U.S. Provisional Patent Application No. 62/127,050 filed on Mar. 2, 2015, which is hereby incorporated by reference in its entirety. Priority is claimed pursuant to 35 U.S.C. § 119.

### STATEMENT REGARDING FEDERALLY SPONSORED RESEARCH AND DEVELOPMENT

This invention was made with Government support under Grant No. 1332275 from the National Science Foundation. The Government has certain rights in the invention.

### FIELD OF THE INVENTION

The technical field generally relates to analyte detection methods and devices. In particular, the technical field relates to analyte detection schemes that utilize entropy-driven catalytic hybridization of DNA oligonucleotides for signal generation and amplification.

### BACKGROUND

In order to detect a target molecule or particle of interest standard ELISA (enzyme-linked immunosorbent assay) or related immunoassay technologies have been developed. These assays start with recognition elements (such as antibodies, aptamers, mini-bodies, etc.) that are conjugated to a surface, bead, or other reaction component. For example, an antibody could be conjugated to a surface or a bead. Next, a sample containing the analyte (e.g., antigen) will be exposed to the first surface (or bead) to bind to the surface bound recognition element. Finally, a secondary recognition element (e.g., antibody) that also binds to the analyte is introduced to form a sandwich structure. The secondary recognition element also is conjugated to a signal amplification element, which often includes an enzyme which catalytically acts on an enzyme substrate to create a colored or fluorescent signal. Alternatively, the secondary recognition element may also be conjugated to include an oligonucleotide sequence that is amplified by a nucleic acid amplification reaction (e.g., PCR). In this case, signal can be generated by an intercalating dye, molecular probes, or other readouts that are indicative of DNA amplification.

Traditional ELISA requires a significant number of wash and sample preparation steps which limits its use in a point-of-care setting and requires significant automation hardware and robotics to perform in batch for example in clinical microbiology laboratories or high-throughput screening. Other conventional assays require surface coating, washing, thermocycling (e.g., PCR), and micro/nano-fabrication. Homogenous immunoassays have also been developed to detect analytes, however, often these assays lack sensitivity because they do not employ signal amplification, or require several separate reagent addition and temperature control steps.

There have been several homogenous (one-pot) assays developed to address these challenges of speed and automation with ELISA. In a homogenous assay, the key challenge is to trigger signal generation only in the presence of analyte. The dominant strategy to achieve specificity relies on forming a sandwich of the analyte between two recognition elements, such that two components in proximity are needed to generate signal. Proximity-induced change in fluorescence resonance energy transfer (FRET) is a good example although detection limits are not satisfactory for many applications due to signal generation without amplification. Signal amplification is achieved in the proximity ligation or extension techniques; however, these successful approaches still require sequential additions of reagents, a set of multiple polymerase activities and/or thermocycling, and long assay times (˜1 hour).

These issues, along with interference when running assays in non-ideal solutions (e.g., blood or plasma) limit the potential of these assays in a diagnostic setting, and thus the current rapid, on-site diagnostic systems mostly rely on lateral flow test kits that operate without amplification and therefore typically possess low sensitivity. For instance, more than ten rapid test kits exist for influenza in the United States, all approved by the US Food and Drug Administration (FDA), which can provide a yes/no answer in around 30 minutes with a limit of detection of only ˜5 μg/mL of a viral antigen nucleoprotein (˜90 nM). Also, the performance drastically varies due to several factors from operation and sample fluid types, which leads to their poor performance with a clinical sensitivity of ˜50% and clinical specificity of ˜90% under optimal conditions.

### SUMMARY

A biomolecular assay platform is described herein that provides for fast, homogenous, and ultrasensitive detection of analytes or other molecular markers that can significantly benefit both clinical diagnostics and research. The platform described herein provides for an affordable, fast, homogenous (one-pot) and ultrasensitive detection platform for analytes. Applicants have named this new assay the “Homogenous Entropy-driven Biomolecular Assay (HEBA)” that overcomes the aforementioned limitations inherent with ELISA-based assays. In HEBA, the presence of the analyte leads directly to the catalytic generation of a signal from released oligonucleotides without other enzymatic ligation or amplification steps. Because no addition of enzyme is necessary to generate the signal, the process can be robust over a variety of conditions (e.g., operation in whole blood or plasma). Also, DNA is more stable over long periods of time and larger temperature ranges than enzymes which allows for a longer shelf life and robust reagents for the assay. HEBA operating in a digital manner can also lead to improved quantification for a homogenous assay.

HEBA can be compared to the gold standard, ELISA, and its derivatives. In essence, HEBA is a fundamentally different scheme to perform the molecular recognition and signal amplification steps that occur in ELISA and can serve in all of the markets where ELISA is also used (e.g., in molecular diagnostics, and research tools). The limiting aspects of ELISA include the need for initial binding to a recognition element to be at a surface or other solid phase (e.g., bead) and the need for several washing steps. The surface coating limits the number of available capturing antibodies and diminishes the performance of antibodies (affinity), while washing can cause an unnecessary loss of signal in addition to increasing assay steps and complexity, which prevents widespread use of ELISA in point-of-care diagnostics and increases assay total labor/automation costs. These limitations lead to a sensing performance of a typical ELISA down to picomolar concentrations with a minimum of an hour assay time with a kit. More recent advanced ELISA schemes achieve a few tens of minutes assay time (commercial rapid ELISA kits), or down to femtomolar sensitivity in conjunction with microfluidic technology or nanoparticles.

However, in HEBA, no wash or immobilization is necessary, thus increasing antibody activity, which significantly improves its performance to˜attomolar sensitivity to streptavidin within 10 minutes. When used in conjunction with microfluidic digital assay formats, HEBA may be used to rapidly (e.g., ˜10 minutes) detect the presence of small numbers of molecules (e.g., tens of molecules). HEBA has also shown its outstanding performance and sensitivity in complex matrices like blood, which is not true in many cases with conventional assays, and thus the HEBA will find its use in a wide range of analytical applications in both research and clinical diagnostics. Also, unlike polymerase chain reaction (PCR), HEBA does not require thermocycling to generate detectable signal and has proven its robustness across temperatures (from 15° C. to potentially up to 35° C.), and thus the HEBA will be well suited for on-site monitoring or point-of-care systems with its enhanced sensitivity and assay speed. The beneficial features of HEBA could lead to its use in identifying rare cells in biofluids like circulating cancer cells. In addition, the HEBA will find its use to facilitate improvements in one of the current state-of-the-art assay techniques, digital ELISA, as it removes the need of washing, which is one practical bottleneck of digital assays.

In one embodiment, a method for detecting an analyte in a sample includes the steps of binding a first catalytic precursor to the analyte at a first epitope and binding a second catalytic precursor to the analyte at a second, different epitope to generate a catalytic complex. The catalytic complex is then reacted with a multiplex molecular substrate to generate a first target molecule and an intermediate substrate bound to the catalytic complex. The intermediate substrate is reacted with a dummy reactant to generate a second target molecule, wherein the reaction further generates a waste molecule containing the dummy reactant and an unbound or free catalytic complex. One or more dye(s) specific to the first target molecule and/or the second target molecule are used which output an optical signal that is detected using, for example, an imaging device. The overall reaction described above has substantially net zero enthalpy and a positive entropy change.

In another embodiment, a method for detecting an analyte in a sample includes the steps of binding a first catalytic precursor to the analyte at a first epitope and binding a second catalytic precursor to the analyte at a second, different epitope to generate a catalytic complex. The catalytic complex is then reacted with a multiplex molecular substrate to catalytically generate one or more target molecules and a waste molecule, the multiplex molecular substrate comprising a plurality of molecular subunits non-covalently bound to each other. An optical signal is detected using, for example, an imaging device, that is generated by one or more dye(s) specific to the one or more target molecules, wherein the overall reaction to form the one or more target molecules and the waste molecule has substantially net zero enthalpy and a positive entropy change.

In one embodiment, a method for detecting an analyte includes binding a first catalytic sequence (CS1) of oligonucleotides to the analyte at a first epitope and binding a second catalytic sequence (CS2) of oligonucleotides to the analyte at a second, different epitope. The analyte bound with the first catalytic sequence (CS1) and second the catalytic sequence (CS2) is then reacted with a multiplexed oligonucleotide substrate (MS) to generate a first target oligonucleotide sequence (TS1) and an intermediate substrate containing the first catalytic sequence (CS1) and second the catalytic sequence (CS2). The intermediate substrate is then reacted with a single stranded oligonucleotide dummy sequence (DS) to release a second target oligonucleotide sequence (TS2) and the CS1 and CS2, wherein the reaction further generates a waste sequence (WS) from the dummy sequence (DS). An optical signal generated by dye(s) specific to TS1 and/or TS2 is detected by an imaging device.

In another embodiment, a method of detecting the presence of an analyte in a sample includes generating a plurality of small sample volumes containing a first catalytic sequence (CS1) of oligonucleotides, a second catalytic sequence (CS2) of oligonucleotides, a multiplexed oligonucleotide substrate (MS) containing a first target oligonucleotide sequence (TS1) and a second target oligonucleotide sequence (TS2), a dummy sequence (DS), and one or more dyes specific to TS1 and/or TS2. The small sample volumes are incubated for a period of time (e.g., several minutes) and then imaged. An optical signal generated within the small sample volumes is detected and used to detect the presence or absence of an analyte. The optical signal may also be used to determine the concentration of the analyte in the sample.

While experimental results disclosed herein describe HEBA assays using streptavidin and influenza A nucleoprotein, it should be understood that the HEBA assays (both analog and digital) are not limited by the type or analyte that is detected. The analyte that is detected by the assay may be organic or inorganic. The analyte may include a biochemical molecule such as protein or the like but it may also include other non-biological species. For example, HEBA assays could be used for environmental testing applications.

### BRIEF DESCRIPTION OF THE DRAWINGS

FIG. 1 illustrates the signal generation scheme used in the entropy-driven DNA catalytic oligonucleotide release. Note that in FIG. 1 the catalytic sequence (CS) is a single stranded oligonucleotide and is not bound to any analyte or substrate.

FIG. 2 illustrates the signal generation scheme used in HEBA according to one embodiment of the invention. As seen in FIG. 2, the catalytic sequence (CS of FIG. 1) is split into two pieces or segments (CS1 and CS2) that are bound to two recognition elements (e.g., biotin or an antibody) that are physically brought together by binding to two epitopes located on an analyte. Although there is a gap between these two split catalytic sequences when bound to analyte, the analyte-bound system acts similar to the intact (non-split) catalytic sequence (CS) of FIG. 1 to catalytically release oligonucleotides that can be detected.

FIG. 3A illustrates the multiplex substrate (MS) including the forward and reverse regions outlined in Table 1 for MS1, MS2, and MS3.

FIG. 3B illustrates the waste sequence (WS) that is produced during the HEBA reaction.

FIG. 4 illustrates the fluorescence signal generation scheme using molecular probes according to one embodiment of the invention. For example, one of the target sequences (TS1, TS2) may bind with a nucleic acid sequence containing fluorophore and quencher pair. The target sequence (TS1, TS2) may preferentially bind and remove the quencher from the double stranded nucleic acid, thereby allowing the fluorophore to fluoresce and act as the signal which can then be detected using standard optical detection techniques.

FIG. 5A illustrates a graph of the fluorescent intensity ratio Iₑₓₚₜ/I_{300nMyde} as a function of time for dye only, CS1 only, CS2 only, CS1+CS2, and CS1+CS2+5 nM streptavidin. In this experiment an oligonucleotide-based fluorescent probe targeting TS2 was used.

FIG. 5B illustrates a graph of the percent increase in fluorescent signal for the CS1 and CS2 pair and the xCS1 and xCS2 pair. Better results are seen for the CS1 and CS2 pair. Reaction condition: 20° C. for 10 minutes with a concentration of streptavidin of 5 nM.

FIG. 5C illustrates a graph of the percent increase in fluorescent signal for various concentrations of streptavidin. The graph illustrates the detection limit and dynamic range of HEBA using a biotin-streptavidin pair. Reaction conditions: 20° C. for 10 minutes. Dotted lines in plots indicate the baseline signal (mean±s.d.)

FIG. 5D illustrates a graph of the percent increase in fluorescent signal for various concentrations of influenza A nucleoprotein (NP). The graph illustrates the detection limit and dynamic range of HEBA for influenza A nucleoprotein (NP) spiked into M5 buffer. Reaction conditions: 20° C. for 10 minutes. Dotted lines in plots indicate the baseline signal (mean±s.d.)

FIG. 6A illustrates one type of device used to generate the fractional volumes for digital HEBA. The device is a droplet-based device whereby droplets are formed containing the assay reaction components and then are collected in a chamber downstream of the droplet formation region. The droplets accumulate within the downstream chamber in a two-dimensional array. The array of droplets within the chamber may then be imaged.

FIG. 6B illustrates a magnified view of a portion of the droplet-based device of FIG. 6A. Illustrated is the intersection of the reaction components with sheath flow inlets that is used to generate the droplets as well as the downstream chamber where the droplets are collected. The two-dimensional array of droplets is illustrated.

FIG. 7A illustrates another type of device that is used to generate fractional volumes for digital HEBA. This embodiment of the device is a two-layer, compression-based device. An inner volume is formed between the two layers and, when brought together in a compression process, forms a plurality of wells that hold discrete, fractionated volumes. The fractionated volumes can be formed in an array which may then be imaged.

FIG. 7B illustrates a magnified view of a portion of the compression-based device of FIG. 6B. An inlet channel is illustrated that leads to the larger region containing a plurality of compartmentalized wells. Each well contains a discrete, fractionated volume in response to compression of the multi-layered device.

FIG. 8A illustrates a perspective view of a well-based device such as that illustrated in FIG. 6B for digital HEBA. Repeating arrays of wells are contained in a PDMS-based substrate that is formed on glass. A single array of wells contains 58×58 wells having a diameter of 15 μm. The “positive” or “ON” dark wells (with analyte) are shown while lighter, grey wells (without analyte) are “negative” or “OFF.” Dimensions are for illustration purposes only and not limiting.

FIG. 8B illustrates schematically the two binary states for each well in the device of FIG. 8A. The upper panel indicates the “OFF” state or readout where there is no or minimal net reaction. The lower panel indicates the “ON” state or readout where there is catalyzed net reaction.

FIG. 8C illustrates representative images obtained from digital HEBA (dHEBA) assays using 4 aM (˜30 molecules, upper image) and 100 aM (720 molecules, lower image) influenza A nucleoproteins (NPs) as analyte.

FIG. 8D illustrates the digital HEBA analysis (experimental “ON” well fraction vs. theoretical “ON” well fraction) of influenza A nucleoproteins (NPs) from 4 aM (˜30 molecules) to 10 fM (72,000 molecules).

FIG. 8E illustrates the combined representation of normal or analog HEBA and digital HEBA performance for indication of influenza A NPs over 8 orders of magnitude.

FIG. 9 illustrates a method of performing an analog HEBA assay according to one embodiment of the invention.

FIG. 10 illustrates a method of performing a digital HEBA assay according to one embodiment of the invention.

### DETAILED DESCRIPTION OF THE ILLUSTRATED EMBODIMENTS

HEBA consists of two parts: specific binding to a target analyte and a signal generation scheme. For specificity, the HEBA utilizes recognition molecules that are specific to an analyte (e.g., antibodies, aptamers, or even potentially other “joining” chemical reactions like click chemistry). Note that there should be at least two separate epitopes (with the same or different molecular structure) located on the analyte that can be bound to by at least two recognition molecules (connected to CS1 and CS2 respectively as described herein). The data described herein includes, as part of one experiment, a biotin-streptavidin pair (with four (4) recognition epitopes) used as the recognition element-analyte pair for demonstration purposes. Another experiment described herein used influenza A nucleoprotein (NP) as an analyte and antibodies against NP as recognition elements for an example of a practical application, although the HEBA platform can be used with other specific recognition elements or molecular targets.

As explained herein, the HEBA platform may be run using small volumes of liquids that contain the sample as well as the reaction components. The volume of fluid depends on the nature of the HEBA assay being performed. For relatively high concentrations of analyte the HEBA assay runs in a so-called “analog mode” whereby the samples are contained in standard sized wells of a multiwell plate (e.g., volumes between about 100 μL to mL scale). For low concentrations of analyte, the HEBA assay runs in a so-called “digital mode” or dHEBA where volumes are typically less than 5 μL. Thus, the term small volume encompasses volumes that range from about 5 μL to several mL.

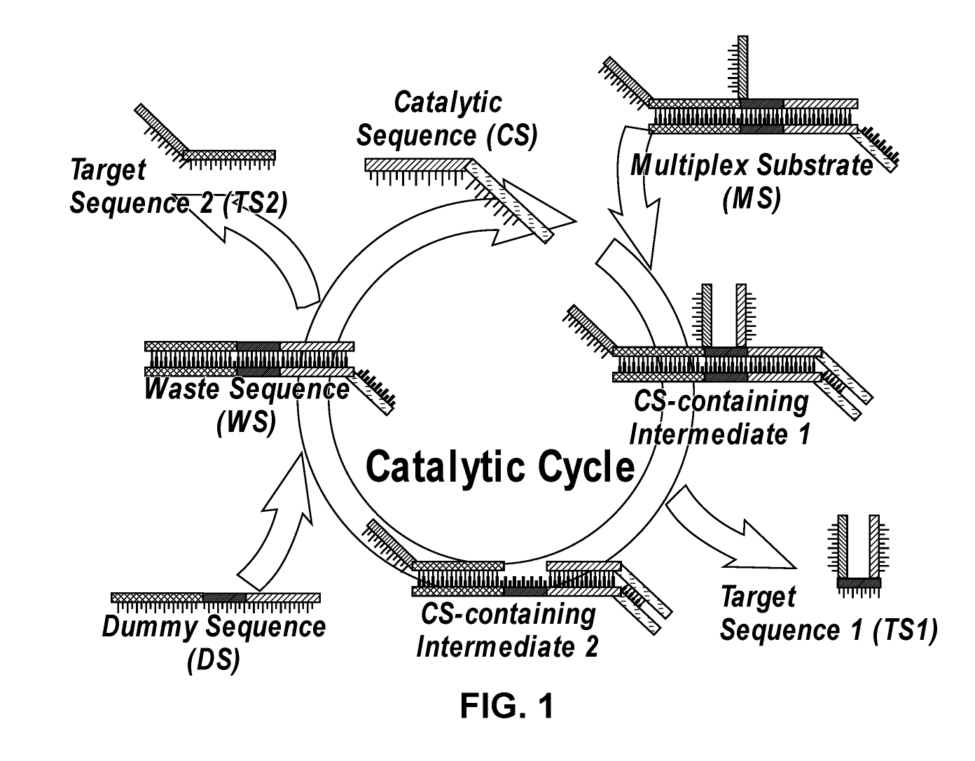

For signal generation and amplification, the HEBA utilizes entropy-driven catalytic hybridization of DNA oligonucleotides. FIG. 1 illustrates the fundamental reaction pathway that forms part of the HEBA process although in FIG. 1 there is a unitary single-stranded catalytic oligonucleotide sequence (CS) that is the target that is recognized. In the HEBA process the CS is not the analyte that is recognized, as explained below, the single-stranded catalytic oligonucleotide sequence (CS) is broken into two sub-units (CS1 and CS2) that are conjugated to recognition elements that bind to different binding sites on the analyte bringing the two sub-units in close proximity so that the two sub-units still exhibits energetically favorable catalytic activity through avidity interactions with the multiplexed oligonucleotide substrate (MS). In short, when a single-stranded catalytic oligonucleotide sequence (CS) and a single-stranded dummy sequence (DS) bind to a multiplexed oligonucleotide substrate (MS) the MS releases two single stranded target oligonucleotide sequences (TS1 and TS2) and the CS back again upon binding and becomes a waste sequence (WS). As a result, the entire reaction becomes a three-reactant-four-product system (MS+CS+DS→CS+TS1+TS2+WS) which can be entropically driven forward when the enthalpy of the system is preserved (or has minimal change). See Zhang et al. Science. 2007 Nov 16; 318(5853):1121-5. Unlike Zhang et al., which detects the presence of the catalytic sequence, in Applicant's HEBA platform, when the reaction proceeds, one can use either or both of the TSs (TS1 or TS2) to generate an amplified signal that is used to detect the presence of the analyte or detect the concentration of the analyte. In HEBA, exposure of the system to analyte generates CS1-analyte-CS2 complex, which in turn can act as CS in the net MS+DS+CS⇄WS+TS1+TS2+CS reaction. Because individual split CS pre-cursors are not capable of accelerating the net reaction, catalytic accumulation of free TS1 and TS2 over time reports the presence of analyte. Note that an advantage of the HEBA platform is that it does not require changing of the reaction components (MS, DS, TS1, TS2) for different analytes, but only the recognition elements bound to the same split CS sequences. It should also be noted, that any enthalpy neutral but entropy positive nucleic acid hybridization/displacement reactions can be used as the signal amplification element in HEBA. For example a reaction with two dummy sequences and three target sequences (MS+CS+DS1+DS2→CS+TS1+TS2+TS3+WS), as long as the number of products is larger than the number of reactants while the enthalpy change of the reaction remains close to zero. Other suitable entropy-driven reactions that are not based on nucleic acids (e.g. based on other non-covalent interactions between components such as protein binding and complex formation), which include a split catalytic element brought into proximity by the analyte to form a catalytic complex could also be used for an amplified readout in the HEBA scheme. Non-covalent interactions could include ionic interactions, hydrophobic interactions, hydrogen bonding, Watson-Crick base-pairing, etc.

In one broad sense, the HEBA platform provides a method for detecting the presence (or in some embodiments concentration of as well as presence) of an analyte in a sample. The method includes binding a first catalytic precursor (e.g., CS1) to the analyte at a first epitope and binding a second catalytic precursor (e.g., CS2) to the analyte at a second, different epitope to generate a catalytic complex. This catalytic complex is then reacted with a multiplex molecular substrate (e.g., MS) to catalytically generate one or more target molecules (e.g., TS1, TS2) and a waste molecule (e.g., WS). The multiplex molecular substrate is formed of a plurality of molecular subunits that are non-covalently bound to each other. As described herein, the multiplex molecular substrate, in one embodiment, comprises oligonucleotides (e.g., MS) that are bound to one another using non-covalent (i.e., Watson-Crick) bonding. The reaction generates one or more unbound target molecules that then interact or bind with dye(s) that generate an optical signal (e.g., fluorescent light) that is detected with an imaging device. The optical signal is used to determine the presence of the analyte in the sample (and in some embodiments the concentration of the analyte). The HEBA platform may also include a dummy reactant (e.g., DS) that is used to generate one or more of the unbound target molecules (e.g., TS2) and re-generate an unbound catalytic complex to allow for catalytic reaction and amplification of signal. The reaction described above has substantially zero net enthalpy change and a positive entropy change. It is the positive entropy change that drives the reaction in the forward direction.

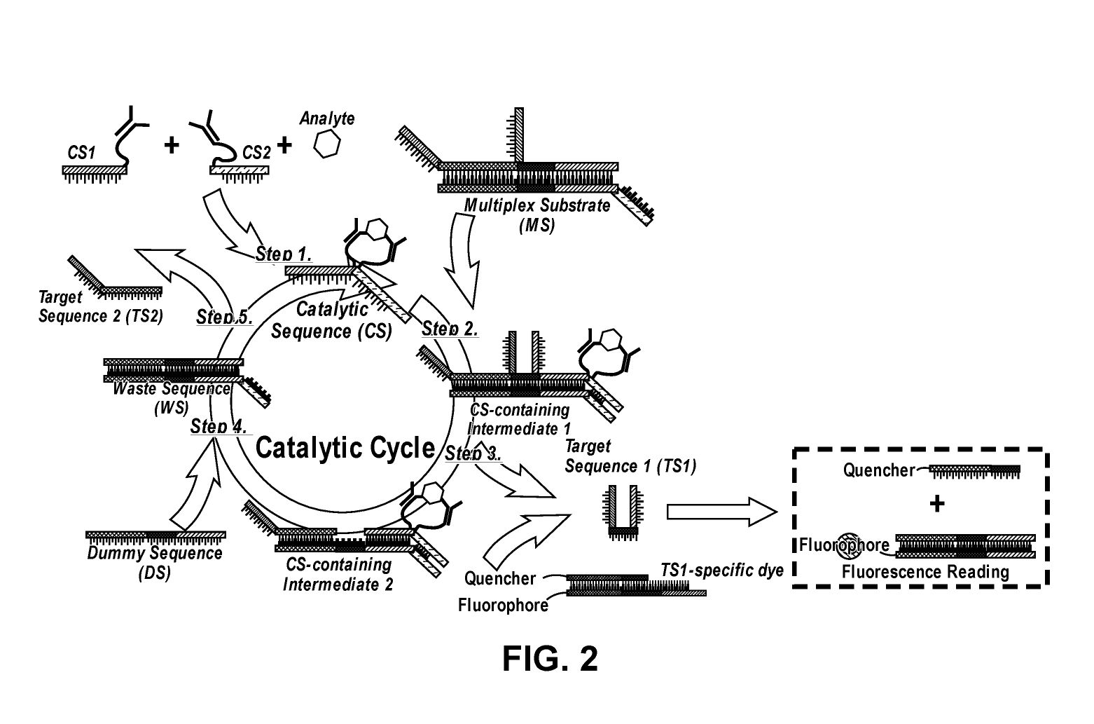

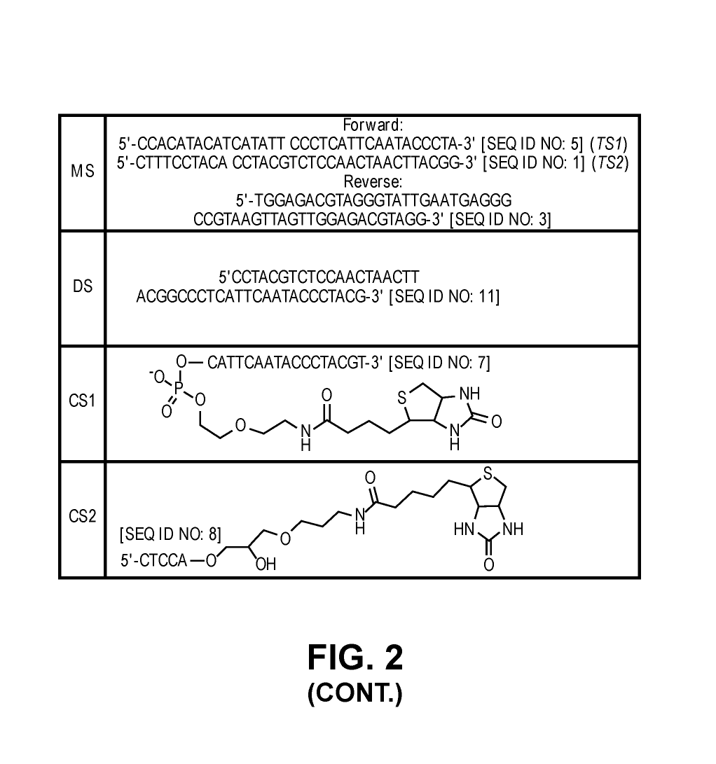

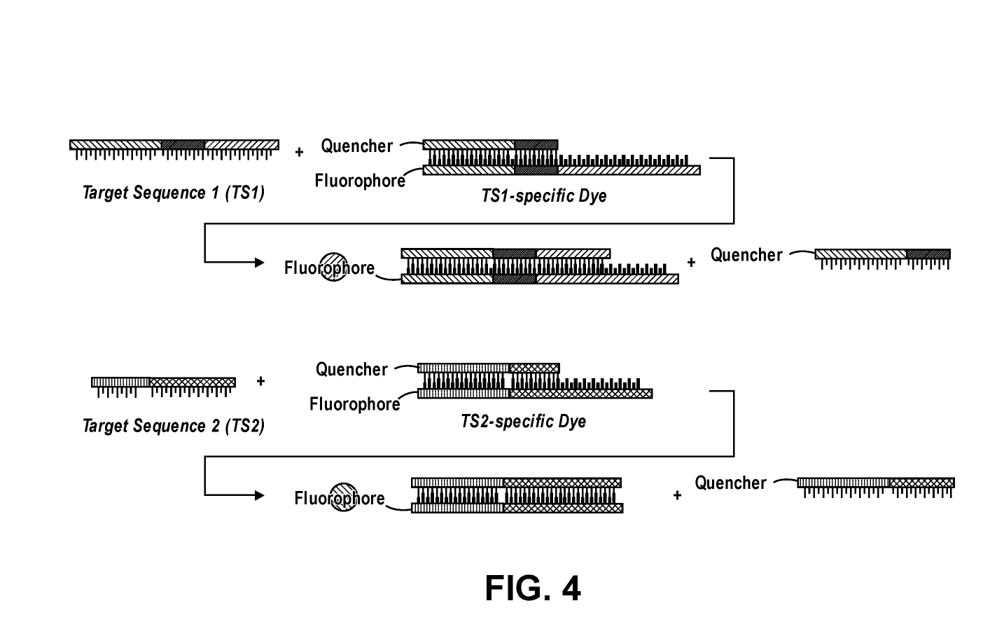

With reference to FIG. 2, the detection scheme is illustrated that is used for both analog HEBA and digital HEBA. In this example, a dye is present that includes an oligonucleotide specific to TS1 (it could also be specific to TS2) contains a fluorophore and a quencher. In the absence of TS1, the quencher molecule inhibits or prevents the emission of fluorescent light from the fluorophore. However, the TS1 competitively binds to the double-stranded sequence and displaces the quencher from the fluorophore, leading to fluorescence signal. While FIG. 2 illustrates a detection scheme whereby TS1 is used to generate a fluorescent reading, the same process can be used for TS2. The dye that is used for TS2 may be different from the dye that is used for TS1 (see FIG. 4). In this regard, different optical signals (e.g., different fluorescent wavelengths) can be generated by TS1 and TS2.

Note that multiplexing of multiple analyte detection reactions can be performed in this format by having separate MS, TS, and recognition element-bound CS oligonucleotide sequences. That is to say, recognition elements for a first analyte bound to a first set of CS fragments contains a different combined nucleic acid sequence compared to a second set of recognition elements specific to a second analyte and so on. Each analyte-specific reaction leads to a separate and specific release of TSs with different nucleic acid sequences. Corresponding complementary TS-specific dye strands with separate fluorophore/quencher pairs are then specifically displaced leading to an analyte-specific fluorescence signal all in a single reaction volume. For example, as explained herein a small volume of liquid (e.g., contained in a droplet or a well) may contain multiple analytes. Because the reactions are analyte-specific and release complementary TS-specific dye strands with separate fluorophore/quencher pairs, a single droplet or well can be analyzed for optical signals in different channels (e.g., optical wavelengths) such that multi-analyte detection in a single small volume is possible.

Alternatively, the conversion of dummy sequence (DS) to a double-stranded WS and concomitant reduction in single-stranded concentration of DS could also be used to generate a signal. For single or multiplexed reactions, alternative readout approaches using nucleic acid specific detection modalities are also possible, including multiplexed readouts (see e.g. Mokany et al. Clin. Chem. 2013 Feb;59(2):419-26, which is incorporated by reference herein). TS and DS sequences could be designed to have active properties (e.g., aptamer or fluorophore binding properties) when in a single stranded form to create a differential signal.

Still referring to FIG. 2, in the HEBA process the presence of analyte links at least two catalytic sequence parts (CS1 and CS2) to drive the catalytic generation of TSs (TS1 (SEQ ID NO: 5) and TS2 (SEQ ID NO: 1)) and fluorescence signal generation , all without any washing steps. For this purpose, the single-stranded catalytic oligonucleotide sequence (CS) of FIG. 1 is split into two pieces; a first single stranded sequence of nucleotides that is a longer chain (CS1 (SEQ ID NO:7)) and a second single stranded sequence of nucleotides that forms a short chain (CS2 (SEQ ID NO:8)). In solution CS1 and CS2 individually cannot efficiently initiate the entire catalytic reaction described above because it becomes energetically unfavorable to displace TSs with CS1 or CS2 alone. An end of each of CS1 and CS2 is conjugated to an analyte-recognition element (e.g., biotin or antibodies). More than one of either CS1 or CS2 can be conjugated to each recognition element for increased probability for reaction with random binding orientations for the at least two recognition elements when forming a sandwich of the analyte. In the experimental demonstration using biotin-streptavidin as the specificity pair both of CS1 and CS2 are biotinylated (5′ end with a 4 carbon chain linker for CS1 and 3′end with a 4 carbon chain linker for CS2) to provide binding specificity to the target analyte, streptavidin. Of course, streptavidin was used for illustration purposes and other analytes may be used provided that the analyte has at least two separate epitopes for binding. As explained herein, influenza A nucleoprotein was used, for example, as the analyte.

It should be understood that the linker length is key to optimize for other binding pairs like antigen-antibody reactions and should be optimized based on the dimensions of the binding pairs. As a result, in the presence of analyte, both catalytic sub-units (CS1, CS2) bound to the analyte form an aggregate assembly of the complete catalytic sequence (CS). Thus, when the catalytic sequence assembly binds it is energetically favorable to displace TSs and move the catalytic reaction forward. Again, the individual pieces (CS1 and CS2) even together in solution but in the absence of streptavidin do not shift the reaction equilibrium substantially. This entire concept of HEBA is illustrated in FIG. 2, and sequence information for all necessary pieces is described in a Table 1 below.

TABLE 1

| Name | Sequence |
|---|---|
| MS1 | Forward: |
| | 5′-CTTTCCTACA CCTACGTCTCCAACTAACTTACGG-3′ [SEQ ID NO: 1] |
| | 5′-CCACATACATCATATT CCCTCATTCAATACCCTACG-3′ [SEQ ID NO: 2] |
| | Reverse: |
| | 5′-TGGAGACGTAGGGTATTGAATGAGGGCCGTAAGTTAGTTGGAGACGTAGG-3′ [SEQ ID NO: 3] |
| MS2 | Forward: |
| | 5′-CTTTCCTACA CCTACGTCTCCAACTAACTTACGG-3′ [SEQ ID NO: 1] |
| | 5′-CCACATACATCATATT CCCTCATTCAATACCCTAC-3′ [SEQ ID NO: 4] |
| | Reverse: |
| | 5′-TGGAGACGTAGGGTATTGAATGAGGGCCGTAAGTTAGTTGGAGACGTAGG-3′ [SEQ ID NO: 3] |
| MS3 | Forward: |
| | 5′-CTTTCCTACA CCTACGTCTCCAACTAACTTACGG-3′ [SEQ ID NO: 1] |
| | 5′-CCACATACATCATATT CCCTCATTCAATACCCTA-3′ [SEQ ID NO: 5] |
| | Reverse: |
| | 5′-TGGAGACGTAGGGTATTGAATGAGGGCCGTAAGTTAGTTGGAGACGTAGG-3′ [SEQ ID NO: 3] |
| CS | 5′-CATTCAATACCCTACGTCTCCA-3′ [SEQ ID NO: 6] |
| CS1 | 5′Biotin-CATTCAATACCCTACGT-3′ [SEQ ID NO: 7] |
| CS2 | 5′-CTCCA-3′Biotin [SEQ ID NO: 8] |
| xCS1 | 5′Biotin-CATTCAATACCC-3′ [SEQ ID NO: 9] |
| xCS2 | 5′-TACGTCTCCA-3′Biotin [SEQ ID NO: 10] |
| DS | 5′-CCTACGTCTCCAACTAACTTACGGCCCTCATTCAATACCCTACG-3′ [SEQ ID NO: 11] |
| TS1 | 5′-CCACATACATCATATTCCCTCATTCAATACCCTA-3′ [SEQ ID NO: 5] |
| TS2 | 5′-CTTTCCTACACCTACGTCTCCAACTAACTTACGG-3′ [SEQ ID NO: 1] |
| WS | Forward: |
| | 5v-CCTACGTCTCCAACTAACTTACGGCCCTCATTCAATACCCTACG-3′ [SEQ ID NO: 12] |
| | Reverse: |
| | 5′-TGGAGACGTAGGGTATTGAATGAGGGCCGTAAGTTAGTTGGAGACGTAGG-3′ [SEQ ID NO: 13] |
| TS1Dye | Forward: |
| | 5′-TET-CCACATACATCATATT CCCT-3 [SEQ ID NO: 14] (TET is Tetrachlorofluorescein dye) |
| | Reverse: |
| | 5′-GTATTGAATG AGGG AATATGATGTATGTGG-3′IowaBlack [SEQ ID NO: 15] |
| TS2Dye | Forward: 5′-ROX-CTTTCCTACA CCTACG-3′ [SEQ ID NO: 16] (ROX is Carboxy-X-Rhodamine dye) |
| | Reverse: 5′-TGGAGA CGTAGG TGTAGGAAAG-3′Iowablack [SEQ ID NO: 17] |

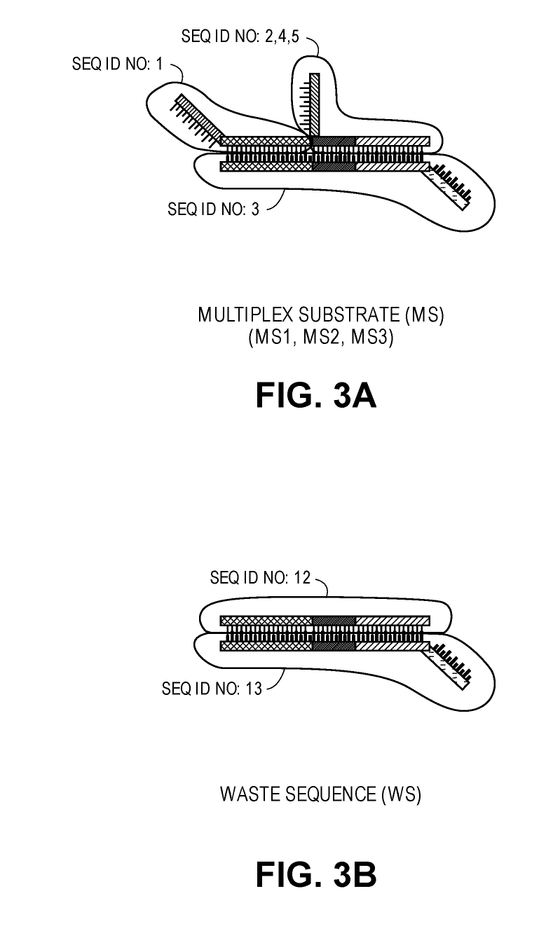

FIG. 3A illustrates the multiplex substrate (MS) including the forward and reverse regions outlined in Table 1 for MS1, MS2, and MS3. FIG. 3B illustrates the waste sequence (WS) that is produced during the HEBA reaction.

Experiment #1 (Biotin-Streptavidin)

HEBA was first demonstrated using biotin-streptavidin as the specificity pair. All DNA oligonucleotides, including biotinylated oligos, used were purchased through custom oligonucleotide synthesis services from Sigma Aldrich (St. Louis, Mo.) and Integrated DNA Technologies, IDT (Coralville, Iowa). Temperature control in this work (including Experiment #2) was performed using an Eppendorf Mastercycler gradient personal cycler (Hauppauge, N.Y.). Fluorescence measurement was performed on an Eclipse Ti-E inverted microscope (Nikon, Melville, N.Y.) operated via NIS elements imaging software (Nikon, Melville, N.Y.).

Necessary DNA oligonucleotide components, multiplex substrate (MS), dummy sequence (DS), catalytic piece #1 and piece #2 (CS1 and CS2), and the dye sequences (either TS1Dye or TS2Dye) were prepared in tris(hydroxymethyl)aminomethane-ethylenediaminetetraacetic acid (Tris-EDTA) buffer (100× stock from Sigma Aldrich) supplemented by the below-specified concentrations of Mg²⁺and/or Na⁺ (Sigma Aldrich, St. Louis, Mo.). The mixing order of the components is important and the order varied except that addition of the analyte, e.g., streptavidin (or NP-spiked samples) was always following the presence of both CS1 and CS2 in the solution. This is to minimize the formation of CS1-analyte-CS1 and CS2-analyte-CS2 complexes that are incapable of driving the entire reaction forward. Concentration of individual components is as follows: MS (MS3 sequence)—300 nM, DS—300 nM, TS1Dye or TS2Dye—300 nM, and CS1 or CS2—30 nM. This set of concentrations is fixed in this demonstration unless specified. All reactions (with the exception of digital HEBA) are at the 250 μL scale in 96 well plates. That is to say, for the analog HEBA experiments, the small volume used in the biotin-streptavidin experiments were deposited into standard sized 96 well plates. Fluorescent imaging results of the wells were obtained using the Eclipse Ti-E inverted microscope as described above.

For biosample solutions, whole blood was obtained from healthy donors by drawing into ethylenediaminetetraacetic acid (EDTA)-coated vacutainer tubes according to the approved IRB protocol ID#11-001120. Whole blood was used as-is for blood control, and for blood samples analyte was spiked into whole blood at defined concentrations. For plasma, fresh whole blood was centrifuged at 3000 rpm for 5 minutes and the supernatant was transferred and used as the control, and for plasma samples analyte was spiked into isolated plasma at defined concentrations.

For antibody biotinylation, antibodies were biotinylated using EZ-link N-hydroxysuccinimide (NHS)-polyethylene glycol 12(PEG12)-biotin conjugation kit from Life Technologies (Grand Island, N.Y.). According to the biotinylation protocol provided by the vendor, individual antibodies are expected to be conjugated with 2˜3 biotin molecules although detailed characterization was not performed.

All data presented herein (including for NP (Experiment #2) are fluorescence intensities measured on an Eclipse Ti-E inverted microscope (Nikon, Melville, N.Y.) operated via NIS elements imaging software (Nikon, Melville, N.Y.) equipped with a CCD camera (QuantEM, Photometrics, Tucson, Ariz.). Unless specified, all numeric values are fluorescence intensity from analyte-containing conditions with respect to fluorescence intensity from control conditions (containing all reaction components, including CS1 and CS2, but no analyte). Threshold levels are based on the fluctuation in control conditions (mean±3 s.d.). The reaction/measurement is at room temperature at 10 minutes unless specified. For dHEBA (Experiment #2), while it is straight-forward to discriminate wells that were lit up (i.e., ON) or not (i.e., OFF), a control set is always run in parallel for confirmation—the threshold level was set to mean±3 s.d. of unlit wells.

Biotinylated antibodies exposed to a stoichiometric excess amount of streptavidin were separately incubated with catalytic pieces (CS1 or CS2) to prepare CS1 (or CS2)-streptavidin-Ab complexes. Once the complexes were prepared, the solution was incubated with excess biotin to quench any remaining binding moieties on streptavidin. This is a necessary step because, while streptavidin alone has no impact on the reaction, there still is a possibility of free CS1 and CS2 pieces, which may be present in CS1-streptavidin-Ab and CS2-streptavidin-Ab solutions, that could form CS1-Streptavidin-CS2 complexes to move the reaction forward when all binding sites of streptavidin are not occupied. The final concentration of antibodies was calculated to be 2.25 μM. When necessary, these CS1- or CS2-antibody solutions were incubated (3 times, 5 minutes) in streptavidin-coated well plates (Life technologies, Grand Island, N.Y.) to remove free biotin, CS1, and CS2.

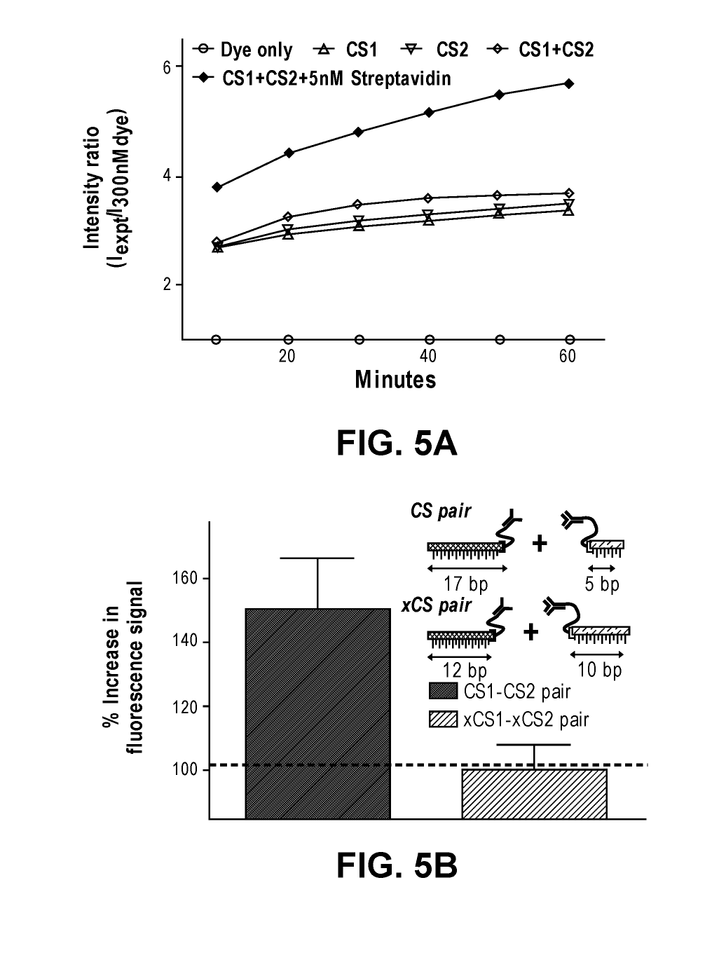

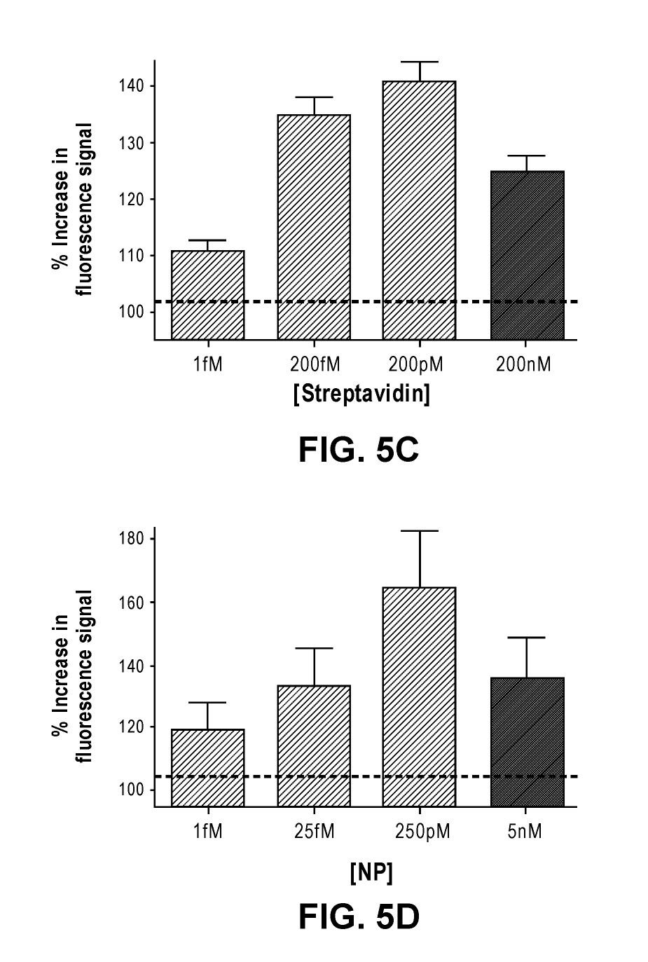

The 5′ end of CS1 and 3′ end of CS2 were biotinylated so that a CS1-streptavidin-CS2 complex could be generated in the presence of streptavidin. The addition of 5 nM streptavidin to the reaction mix led to a fluorescent signal that was larger than CS1 or CS2 alone, and CS1 and CS2 together without streptavidin, demonstrating that CS1-streptavidin-CS2 acts as a single catalytic element to accelerate the net reaction as seen in FIG. 5A. To note, reagent mixing order played a role in the HEBA performance - addition of analyte (streptavidin) following both CS1 and CS2 was critical for its success, otherwise CS1-streptavidin-CS1 or CS2-streptavidin-CS2 complexes can be generated at higher rates leading to assay performance variation.

The MS has negligible direct interaction with TS-specific probes due to their sequence, and thus, the baseline fluorescence signal in the control wells is originated from the natural dissociation of TSs from MS and the released TSs open up their respective probes. With the rate constant for “MS→TS1+TS2” to be k_{MS}=2.3×10 M⁻¹s⁻¹, and the rate constant for a “TS+Dye→Fluorescence” interacting with the dye probe to release the fluorophore to be k_{TS}=4.0×105 M⁻¹s⁻¹ as studied in a separate study, 8.9 nM fluorophore contributes to the baseline signal at a 10-minute time point. With this said, a 110˜120% signal increase suggests that 1 fM of analyte has resulted in the release of an additional ˜1 nM fluorophore in the system (FIGS. 5C and 5D (for (NP)). To note, this estimation might be too coarse considering the fact that the existence of CS1 and CS2 in the system can also contribute to the baseline signal by slightly driving the release of TSs from MS without the presence of analyte. As such, the analyte signal (CS1+CS2+Analyte) was normalized with respect to this control fluorescence signal (CS1+CS2).

Using the streptavidin-biotin system, the catalytic complex that would maximize signal generation above background was explored. Two split points were compared in the catalytic sequence (Table 1 and FIG. 5B): a CS1-CS2 pair, in which CS1 had both toehold binding and branch migration functionality to displace TS1, and a xCS1-xCS2 pair in which xCS1 has no toehold region to localize it prior to replacing the complementary portion of TS1. While CS2 (xCS2) localizes CS1 (xCS1) close to the MS backbone, designing CS1 such that it contains a toehold binding region was favored for optimal HEBA performance. In the CS1-CS2 scheme, a two base pair toehold region in CS1 acts to localize CS1 on the MS backbone and accelerate the displacement of TS1. For the same reason, changes in the MS sequence at the CS binding region leads to changes in the HEBA performance. The best HEBA performance was observed with MS3 among MS1, MS2, and MS3, which has an additional two (2) base pair-long toehold binding region for CS1 compared to MS1.

In addition, using this model of the HEBA system the optimal temperature and time scale of operation was characterized. HEBA operated over a range of moderate temperatures (15° C.-35° C.) and a signal increase was found with respect to control (no-analyte) conditions beyond the mean+s.d. within 10 minutes. Although a sufficient signal is generated within 10 minutes, at this time point the reaction has not gone to completion. Signal increases further over longer times, but signal-to-background may not increase substantially beyond 10 minutes. Thus, in the HEBA platform described herein, incubation times may be on the order of several minutes, e.g., about 10 minutes. Additional incubation time beyond the 10 minute mark may also be used to increase the level the signal (e.g., fluorescent light) that is generated.

Next, the impact of stoichiometry of the reactants on HEBA performance was investigated. The assay requires an analyte sandwiched by CS1 and CS2 to form the catalytic complex (CS1-analyte-CS2). Therefore, if the analyte concentration is too high compared to the [CS1] or [CS2] the probability of creating a CS1-analyte-CS2 complex diminishes, and analyte-CS1 or analyte-CS2 complexes become dominant. Obtained data were consistent with such an effect - fluorescence signal diminished for high streptavidin concentrations (200 nM in FIG. 5C). Previous work reported that an optimal stoichiometry to achieve a biotin-streptavidin-biotin complex is ˜10:1 biotin:streptavidin, which is consistent with the observations. The stoichiometry between [Analyte] and [CSs] is also a parameter to be adjusted for HEBA, depending on the expected analyte concentration within an assay.

Because of the amplified nature of the readout, HEBA identified the presence of streptavidin down to 1 fM in Tris-EDTA (TE) buffer with 6˜7 orders of magnitude in dynamic range (FIG. 5C) in a 10 minute 250 μL-scale reaction. Since HEBA amplification is non-enzymatic, the detection limit was not substantially reduced in body fluids, achieving 5 fM in plasma and 50 fM in whole blood with a similar dynamic range.

Experiment #2 (Influenza A Proteins)

A HEBA platform was also developed to detect influenza A nucleoproteins. The purpose of this experiment was to validate the platform for a point-of-care diagnostic assay. As noted herein, current influenza testing kits can provide a yes or no answer within about 30 minutes but have a limit of detection of only around ˜5 μg/mL. Influenza A virus nucleoprotein (NP) was chosen as the target analyte because it is a main conserved protein stabilizing the viral RNA strand and serves as the target for most lateral flow point of care influenza assays. HEBA performance was evaluated with full length NPs in M5 buffer, which is the universal transport media used in clinical microbiology laboratories to store suspected influenza samples. The HEBA assay for influenza A was conducted first using analog HEBA for high concentrations of influenza A. Lower concentrations of influenza A were tested using a digital HEBA platform as described herein.

Monoclonal influenza A nucleoprotein (NP) antibodies were purchased from SouthernBiotech (Cat. # 10770-01 and 10780-01, Birmingham, Ala.). While the vendor provides data concerning the performance of antibodies in binding to NP, the binding to influenza A NPs were confirmed in our lab using full-length NP protein (Novus, Littleton, Colo.) by sandwich ELISA.

Necessary DNA oligonucleotide components, multiplex substrate (MS), dummy sequence (DS), antibody conjugated catalytic piece #1 and piece #2 (Ab-CS1, and Ab-CS2), and the dye sequence (either TS1Dye or TS2Dye) were prepared in Tris-EDTA buffer supplemented with 12.5 mM Mg²⁺ (Sigma Aldrich, St. Louis, Mo.). Again, mixing order of the components varied except for analyte nucleoprotein, which was added after both Ab-CS1 and Ab-CS2. Concentration of individual components is as follows: MS—300 nM, DS—300 nM, TS1Dye or TS2Dye—300 nM, and Ab-CS1 or Ab-CS2—30 nM. All reactions were performed at 250 μL scale in 96 well plates for analog HEBA. Analyte samples were prepared in universal transport M5 buffer by spiking with varying concentrations of nucleoproteins and control samples consisted of the same buffer without NP.

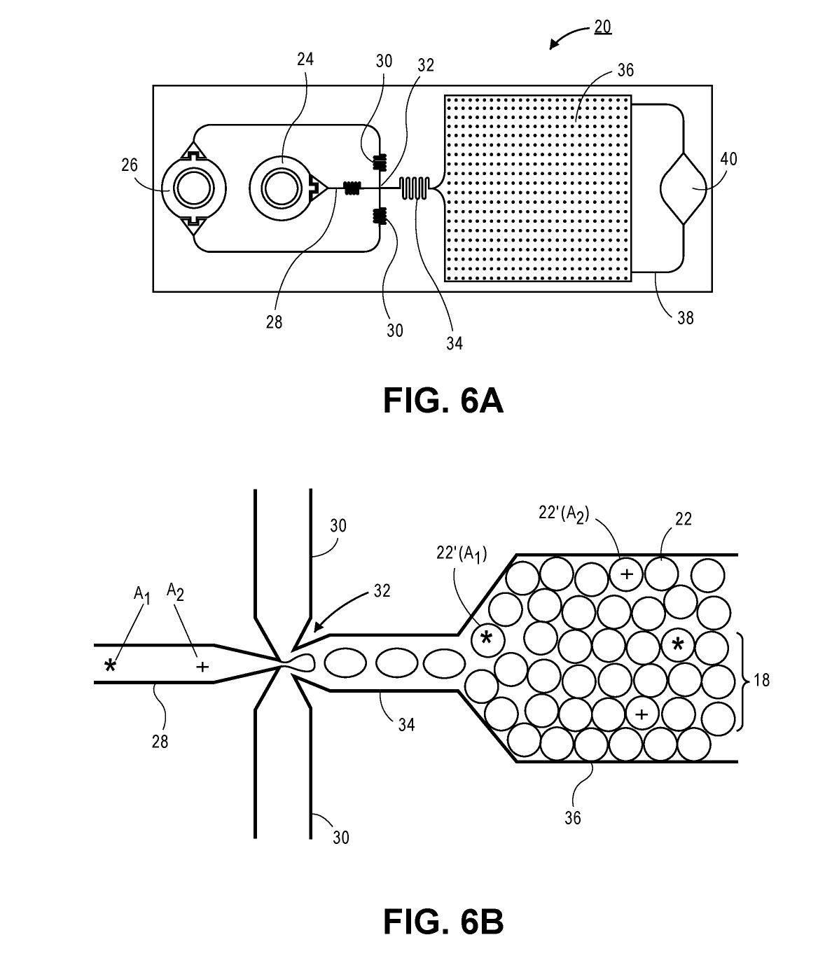

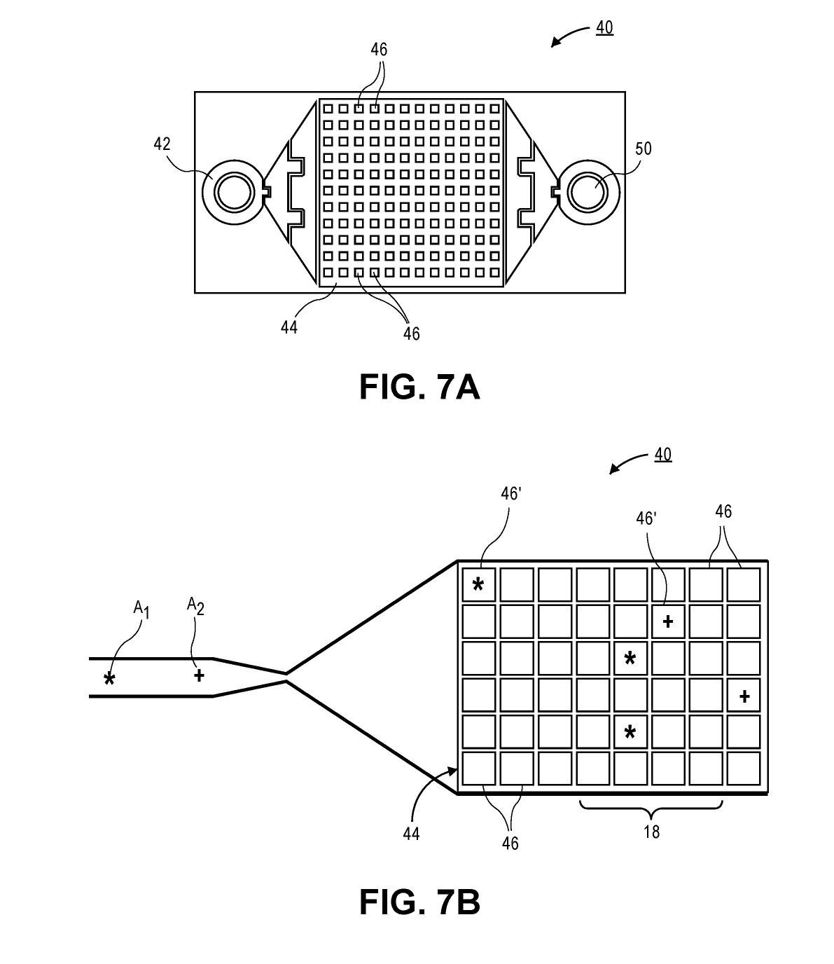

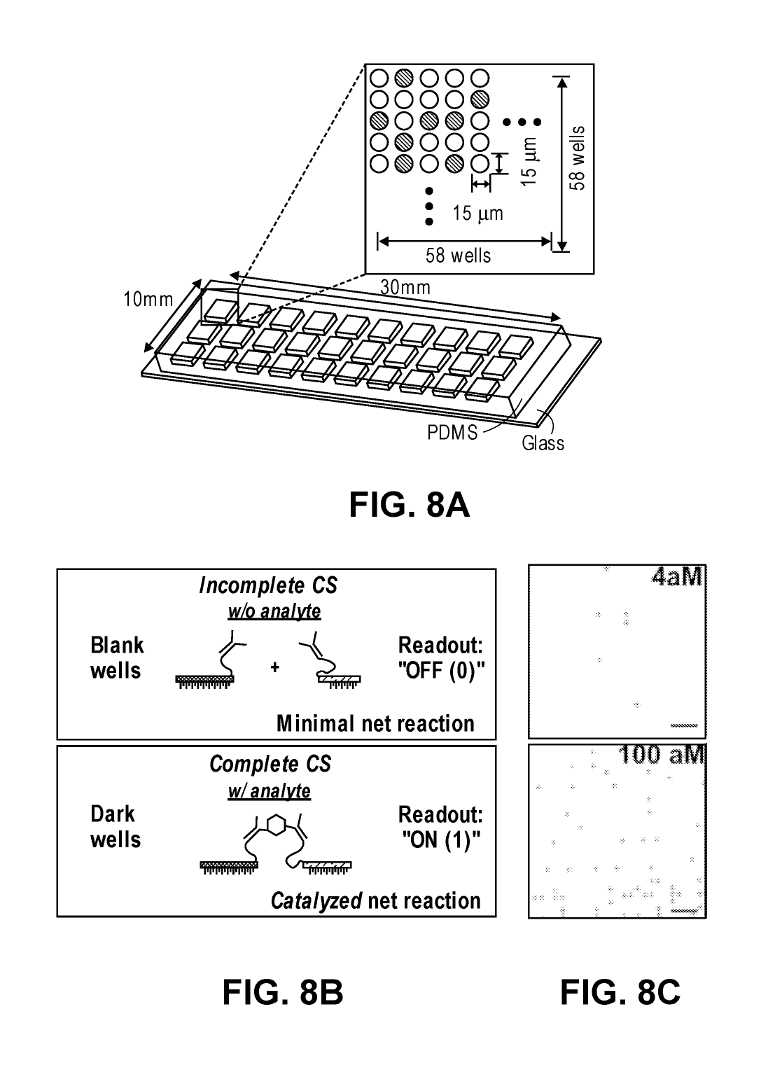

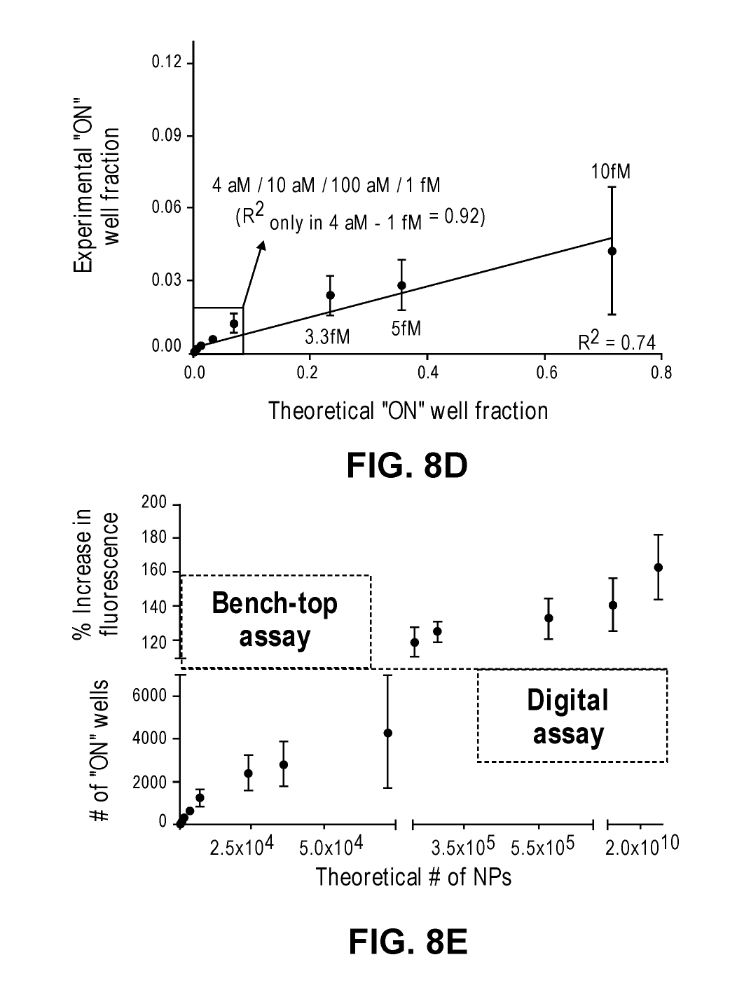

For digital HEBA (dHEBA), the assay platform, which included an array of pL-sized wells such as that described in FIGS. 6A, 6B, 7A, and 7B, was fabricated by standard photolithography techniques in the California NanoSystems Institute at the University of California, Los Angeles. Well dimensions were 15 μm in diameter and 15 μm in height which results in ˜2.65 pL volume for each of 100,920 individual wells (FIG. 8A). Arrays are designed as hierarchical 10×3 arrays of 58×58 2.65 pL wells, with each of the 58×58 arrays designed to be within a ˜2 mm×2 mm field of view of the microscope. Experiments for dHEBA were conducted in an identical fashion as analog HEBA on a well plate except that the PDMS is UV-exposed (or oxygen plasma-treated) to facilitate wetting of the wells by HEBA reaction solution as well as bonding to the glass bottom substrate to firmly compartmentalize the reaction solution into the wells.

With respect to analog HEBA, the platform was able to detect NP down to 1 fM within 10 minutes per test at room temperature as seen in FIG. 5D. For digital HEBA, the platform was able to detect NP down to tens of molecules. There are several design rules that should be considered in developing the molecular machinery for HEBA assays. In order to achieve analyte-dependent generation of an amplified signal in diverse body fluids, specificity of the complex (CS1-analyte-CS2 complex) in generated signal is paramount. Particularly in body fluids such as blood or plasma, there are myriad potential interfering components that can suppress/enhance specificity-determining binding events leading to either false positive or negative signals. As with the approach to increase specificity in sandwich ELISA, in HEBA, antibodies are designed to target different epitopes on the analyte rather than one targeting the Fc or other regions of the primary antibody. This minimizes potential interference from cross reaction due to endogenous/structural similarities for the primary antibody or heterophile antibodies. Despite the sandwich format, current HEBA is somewhat sensitive to non-specific interference from sample matrices. Signal in buffer still is higher by a factor of 110˜130% and 150˜170% when compared to undiluted plasma and whole blood, respectively. Besides interfering immunoglobulin, blood cells and cell debris in blood, can increase the noise in accurate fluorescence readings. Many of these issues could be solved through sample dilution because HEBA shows promising sensitivity in identifying the presence of analyte at low concentrations. Dilution could be considered a more reliable option for HEBA than other methods to handle matrix effects given that many analytes will be present at levels much higher than hundreds to thousands of molecules needed for example with digital HEBA for accurate detection.

The use of DNA-based molecular machinery, rather than enzymes, for amplified signal generation results in a second design rule focused on use of “short” DNA oligonucleotides. The use of DNA oligonucleotides is beneficial in designing a molecular machinery due to its potential circuit diversity that can be simply controlled by Watson-Crick base pairing. While longer oligonucleotides when hybridized will provide better stability in this state under diverse circumstances, short oligonucleotides were chosen not only to seek the best potential to maintain stability but also respond quickly to changes in catalyst complex concentration. As such, all “active” zones for hybridization/displacement on oligonucleotides were over relatively short lengths (less than 30 bps) with minimal chance of secondary structures. Secondary structure can lead to molecular machinery and displacement processes that are more dependent on factors such as ionic strength changes. HEBA performed stably in identifying the presence of analyte over a wide range of salt concentrations (6 mM and 12.5 mM Mg²⁺ with/without 0˜300 mM Na⁺ in TE buffer).

One last key aspect for HEBA is the design of the analyte-containing catalyst that is formed upon sandwich recognition of analyte. As pictured in FIG. 2, the catalyst, CS1-Analyte-CS2, in HEBA is unlike the catalyst in the proximity ligation assay, where two nucleotides in close proximity are ligated to form a single signaling molecule. In HEBA, the two separate oligonucleotides without ligation act to bind and displace in a cooperative manner because of their proximity (and most likely multivalency due to the streptavidin backbone used). The design rule herein was to consider the binding process of the catalyst as a 3-step process (toehold binding-toehold binding-branch migration), not a 2-step process (toehold binding-branch migration). As seen in FIG. 5B, the CS pair is designed to achieve cooperativity in toehold binding; the short CS2 toehold binds to MS providing CS1-Analyte-CS2 initial stability on the MS so that the longer CS1 has time to interact with MS and TS1 for toe hold binding followed by the higher activation energy process of branch migration.

FIGS. 6A, 6B, 7A, and 7B illustrate two different devices that may be used to perform digital HEBA. FIG. 6A illustrates one type of device 20 used to generate the small volumes 18 (illustrated in FIG. 6B). In this embodiment, the device 20 is a microfluidic device that generates micro-emulsions (i.e., droplets 22) that act as the small volumes 18. The droplets may have a variety of diameters but are typically greater than 10 μm. The device 20 includes a first inlet 24 that is used to deliver a sample with the analyte(s) (e.g., A₁, A₂) as well as the reaction mixture that contains the MS, CS1, CS2, DS, and dye(s). Typically, this fluid is an aqueous fluid. A second inlet 26 is provided that can be used to deliver a fluid that is immiscible with the fluid delivered via the first inlet 24. For example, an oil-based fluid may be delivered to the second inlet 26. Fluid may be delivered to the first inlet 24 and second inlet 26 via respective fluid pumps (e.g., syringe pumps or the like; not shown). A channel 28 coupled to the first inlet 24 and intersects with two opposing channels 30 connected to the second inlet 26. The intersection of the channels 28, 30 forms a droplet generation region 32 whereby the aqueous droplets 22 are pinched off using a sheath flow from the opposing channels 30. Note that multiple channels 28 (not shown) may be used to add the various components. In this regard, multiple inlet or input channels can be used to ensure that the various HEBA reaction components are added in the correct order and just prior to droplet formation. Another channel 34 connects the droplet generation region 32 to a downstream droplet collection region 36. The droplet collection region 36 is a three dimensional chamber with a large width and length but small height so that droplets 22 form a two-dimensional array within the droplet collection region 36. The droplet collection region 36 is constructed with a height so that the droplets 22 do not stack in multiple layers which would interfere with the imaging of the discrete, small fractional volumes 18 which is needed for detection and concentration determination in the HEBA assay. The droplet collection region 36 is coupled via channel 38 to an outlet 40 whereby the droplets 22 fluids and like can be removed from the device 20.

FIG. 6B illustrates a magnified view of the droplet generation region 32 and the downstream droplet collection region 36. Channel 28 shows multiple analytes A₁, A₂ in the inlet channel although it should be understood that only a single analyte may be used with digital HEBA. Droplets are pinched off in the droplet generation region 32 and collected in the droplet collection region 36. Certain droplets 22′ contained within the droplet generation region 32 exhibit a positive optical signal because they contain the analyte (e.g., A₁, A₂) and the HEBA reactants MS, CS1, CS2, DS, and dye(s). For example, some droplets 22′ are positive for analyte A₁. These may be identified by emitted fluorescent light of a certain color or wavelength (or range of wavelengths). Likewise, other droplets 22′ are positive for analyte A₂. These may be identified by emitted fluorescent light of a different color or wavelength (or range of wavelengths). The optical signal is binary in that each droplet either emits a positive optical signal (i.e., ON state) or not (i.e., OFF state). While FIGS. 6A and 6B illustrate one mechanism of droplet formation using sheath flow to pinch off droplets it should be understood that other droplet-forming devices and systems can be employed to generate droplets that contain a sample and HEBA reactants.

FIG. 7A illustrates another embodiment of a device 40. In this embodiment, the small volumes 18 (as seen in FIG. 7B) are created using a plurality of discrete wells 46 which are formed in the device 40. Some of the wells 46′ contain small volumes 18 that exhibit a positive optical signal because they contain the analyte (e.g., A₁, A₂) and the HEBA reactants MS, CS1, CS2, DS, and dye(s). With reference to FIGS. 7A, the device 40 includes an inlet 42 that is fluidically connected to a region 44 holding a plurality of wells 46. The wells 46, in one embodiment, are formed in a flexible layer 48 which may include a flexible substrate such as polydimethylsiloxane (PDMS) that is adhered to an optically transparent substrate such as glass. Each well 46 forms a small, fractional volume 18 such that when the device 40 is fully assembled and loaded with fluid it forms an array with discrete, separate reaction volumes. The size of the wells 46 may vary but wells 46 having a width of around 15 μm and a height of 15 μm may be used as disclosed herein. The device 40 further includes an outlet 50 whereby fluids can be removed from the device 40. The device 40 is a two-layer device that is formed from by compressing the flexible layer (e.g., PDMS) containing the wells 46 against an optically transparent substrate (seen in FIG. 8A). The optically transparent substrate may include a glass slide although other optically transparent materials may be used (e.g., plastics, etc.). During use, the two-layer device 40 is filled with the assay components that include the sample which may include the analytes (e.g., A₁, A₂) and the HEBA reactants MS, CS1, CS2, DS, and dye(s) until all the wells 46 are filled and all dead volume is gone. There exists a small height in all areas outside the wells 46 to ensure complete fractionalization once the device 40 is compressed. Note that multiple inlet or input channels (not shown) may optionally be used to add the various components. In this regard, multiple inlet or input channels can be used to ensure that the various HEBA reaction components are added in the correct order.

FIG. 8A schematically illustrates a region of the wells 46 in a device of the type disclosed in FIGS. 7A and 7B. As seen in FIG. 8A, certain wells 46 (dark colored wells 46) exhibit a positive or ON signal. FIG. 8B illustrates how the positive wells 46 exhibit a readout of ON or (1) with a catalyzed net reaction due to the presence of the analyte. Conversely, the negative wells 46 exhibit a readout of OFF or (0) with minimal net reaction due to the absence of the analyte. FIG. 8C illustrates representative fluorescent images of the wells 46 obtained using 4 aM (˜30 molecules) and 100 aM (720 molecules) influenza A nucleoproteins (NPs) as analyte. The higher concentration results in a larger number of wells 46 that emit a positive or ON signal.

FIG. 8D illustrates a graph illustrating the digital HEBA results of influenza A nucleoproteins (NPs) at a concentration range from 4 aM (˜30 molecules) to 10 fM (72,000 molecules). The dHEBA experimental results were obtained using a well-based device like that illustrated in FIG. 8A. As seen in FIG. 8D, the results were substantially linear for NP concentrations between 4 aM and 1 fM (R²=0.94 for 4 aM-1 fM) while the response started to deviate from linearity at concentrations >1fM. Deviation is expected for digital assays, because when assuming a Poisson distribution of molecules into the segmented volumes, the probability of a single NP per element decreases with increasing analyte concentration. The probability of wells with more than one analyte molecule can be calculated by the following equation:

$$p = 1 - λ - \frac{( {e}^{- l n λ} \times {( - ln λ )}^{k} )}{k !} where k = 1 \qquad Eq . 1$$

where λ represents the empty well ratio. Table 2 below is a probability table that includes the probability that wells will have more than one analyte molecule contained therein.

TABLE 2

| | Empty | | | | |
|---|---|---|---|---|---|
| | Well | Probability | | Empty | Probability |
| [NP] | Ratio | (#)* | [NP] | Well Ratio | (#)* |
| 4 aM | 1.00 | 0.00 (0.00) | 1 fM | 0.93 | 0.00 (8.77) |
| 100 aM | 0.99 | 0.00 (0.00) | 3.3 fM | 0.76 | 0.03 (101.55) |
| 200 aM | 0.99 | 0.00 (0.34) | 5 fM | 0.64 | 0.07 (245.30) |
| 500 aM | 0.96 | 0.00 (2.17) | 10 fM | 0.29 | 0.36 (1195.20) |

*Probability (#) indicates probability (number) of wells with more than one analyte molecule

As seen above, the number of wells expected to have two or more NP molecules becomes greater than one at NP concentrations of 500 aM in the current dHEBA platform, which supports observations of dHEBA performance deviating from linearity within the 500 aM˜1 fM range.

FIG. 8E illustrates the broad dynamic range for the identification of influenza A NPs that is obtained when one considers both analog HEBA as well as digital HEBA. Digital HEBA is best suited for low copy numbers of NPs and this is seen in the bottom portion of the graph of FIG. 8E. Recall that for digital HEBA the number of “ON” wells is used to measure the presence of the NPs as well as determine the copy number. Analog HEBA is best suited for large copy numbers of NPs which is seen on the upper portion of the graph of FIG. 8E. For analog HEBA, rather than look at the number of “ON” wells one typically looks to the % increase in fluorescence. Taken together, both analog and digital HEBA provide for the identification of NPs at concentrations that range over eight (8) orders of magnitude.

As seen in FIGS. 8D and 8E, the digital implementation of HEBA, despite a narrow dynamic range due to the limited number of wells, provided better quantitation performance compared to its analog version. Considering the fact that a common clinical practice is to split samples for multiplexed examination, and that dHEBA consumes a very small volume of sample (few μL) while achieving sensitivity approaching the single-molecule level, it will be of interest in achieving improved quantitation. This opens additional potential of dHEBA in splitting samples for dilution to reduce matrix effects and/or multiplexed examination for multiple analytes or markers. For example, a sample containing analytes could be diluted and then split into separate volumes that had separate recognition element pairs (bound to CS1 and CS2) specific to different analytes in the sample. The diluted samples with HEBA reagents could then be input into different regions of an array of segmented volumes or separate arrays of droplets to perform multiplexed dHEBA

Table 3 below illustrates the comparison of the HEBA platform described herein with various other assays.

TABLE 3

| | Typical | Amplification | | | |
|---|---|---|---|---|---|
| | Target | Power* | | Procedure | |
| Assay | Analyte | (S/N^{†}) | Sensitivity | (Thermal Control) | Assay Time |
| BiFC^{‡} | Protein | No amplification | nM | Simple | min |
| | | | | (no) | |
| Molecular Beacon | Nucleic Acid | No amplification | nM | Simple | min |
| | | | | (no) | |
| FRET/BRET^{§} | Protein | No amplification | nM | Simple | min |
| | | | | (no) | |
| Biobarcode | Nucleic Acid | No amplification | nM~sub-nM | Moderate | min |
| | | | | (no) | |
| Proximity Ligation | Protein | ~10⁶ | pM~sub-pM | Moderate | hr |
| | | (~10) | | (minimal^{¶}) | |
| PCR | Nucleic Acid | 2^{n#} | Single-molecule | Intensive | hr |
| | | (10~100) | | (yes) | |
| ImmunoPCR | Protein | 2^{n#} | Single-molecule | Intensive | hr |
| | | (10~100) | | (yes) | |
| HEBA | Protein | ~10⁶^{\|\|} | fM | Simple | ~10 min |
| | | (~2) | | (no) | |

*Number of signaling molecules generated compared to the number of analyte molecules
^{†}Ratio of signal increase compared to baseline control
^{‡}Bimolecular Fluorescence Complementation
^{§}FRET: Főrster resonance energy transfer, BRET: Bioluminescence resonance energy transfer
^{¶}Depending on application, but typically isothermal
^{#}n = the number of cycles, typically ~40 leading to an amplification power of ~10¹²
^{||}Assuming the control to be all reagents with no TS

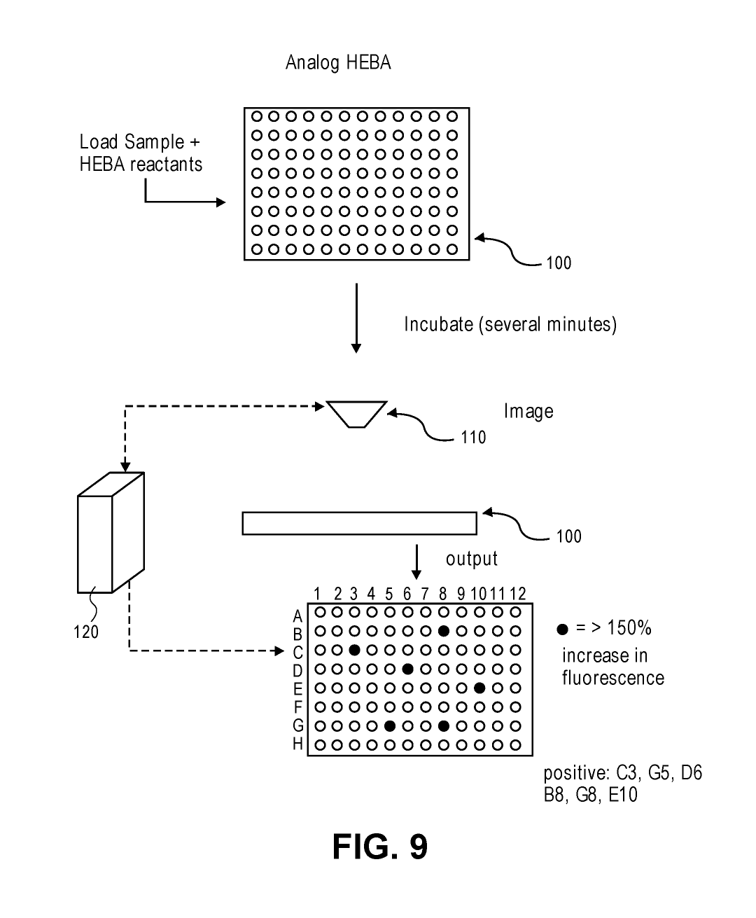

FIG. 9 illustrates an embodiment of the analog HEBA assay. In this embodiment, samples (which may or may not contain the analyte or target of interest) are loaded into a sample holder 100. The sample may include a clinically relevant solution such as buffer, plasma, whole blood, semen, pleural fluid, and the like. In some situations, the sample may be diluted prior to addition. The sampleholder 100 may include a standard well plate such as a 96 well plate as illustrated in FIG. 9. Sample and HEBA reagents are loaded into respective wells (volumes of about 100 μL to mL scale) of the sampleholder 100 using manual or automated loading techniques known to those skilled in the art (e.g., pipette, etc.). The sample and HEBA reactants need to be loaded into the wells in a specific order. For example, the MS and DS with the dye(s) are loaded into the wells followed by CS1 and CS2 together; followed finally by the addition of the sample. Alternatively, the sample can be added to MS, DS and dye(s) followed by the addition of CS1 and CS2. CS1 and CS2 need to be added together and either before or after addition of the analyte (e.g., sample). After the sample and HEBA reagents are loaded in the sample holder 100, the sampleholder 100 is incubated for several minutes (e.g., at least 5 minutes and more preferably around 10 to 15 minutes). Longer incubation times will produce a stronger fluorescent signal as the reaction progresses even further in a linearly catalyzed fashion. Incubation can take place at or near room temperature as there is no need for thermocycling and the HEBA reaction functions over a wide range of temperatures. Next as seen in FIG. 9, the sampleholder 100 is then imaged with an imaging device 110. The imaging device 110 is able to detect fluorescent radiation that is emitted from the wells contained in the sample holder 100. The imaging device 110 may include a conventional fluorescent imaging microscope or imager or it may even include a lens-less imager that have a wide field of view such as the mobile based fluorescent imager disclosed in U.S. Pat. No. 9,057,702, which is incorporated by reference herein.

The imaging device 110, regardless of the type, obtains images of the individual wells of the sample holder 100. FIG. 9 also illustrates a computing device 120 that may be associated with the imaging device 110 or separate therefrom that may be used process the raw image files obtained from the imaging device 110. The computing device 120 may calculate the relative intensity of the wells (based from a baseline) and can also map particular image locations to dedicated well locations. In those wells that contain the analyte or other target, the HEBA reaction is driven forward and effectuates the production of fluorescent light from one or more dyes contained therein. The intensity of the fluorescent light is obtained from images acquired by the imaging device 110 and the location of each well is known. In this particular experiment, those wells deemed “positive” for the analyte or target are those wells where the measured fluorescence level has increased above 150% above a baseline value. Of course, other “positive” cut-off thresholds may be used. In the example of FIG. 9, wells at C3, G5, D6, B8, G8, and E10 are positive. In analog HEBA the intensity may be used not only to detect the presence of the analyte or target but it may also be used to calculate or estimate the concentration of the analyte or target in a sample. For example, by using the measured intensity level as well as knowing the elapsed reaction time, calibration curves or the like may be used to translate the measured intensity level into a concentration reading.

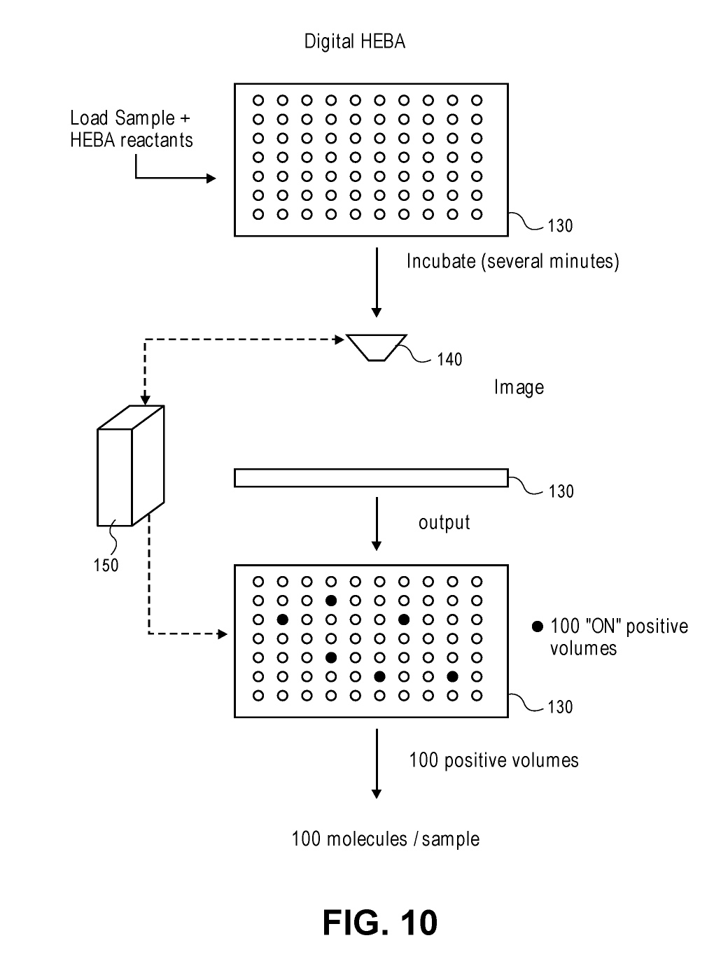

FIG. 10 illustrates an embodiment of the digital HEBA assay. In this embodiment, samples (which may or may not contain the analyte or target of interest) are loaded into a microfluidic sample holder 130. The sample may include a clinically relevant solution such as buffer, plasma, whole blood, semen, pleural fluid, and the like. In some situations, the sample may be diluted prior to addition. In fact, dilution of the sample in digital HEBA is useful to avoid matrix effects and can also be used when digital HEBA is used for multiplexed assays of multiple analytes or targets. FIG. 10 illustrates a microfluidic device 130 such as that illustrated in FIGS. 7A, 7B, and 8A where small (<100 μL), fractionated volumes are created that hold the sample and HEBA reactants.

The sample and HEBA reagents need to be loaded into the wells in a specific order. For example, the MS and DS with the dye(s) are loaded into the wells followed by CS1 and CS2 together; followed finally by the addition of the sample. Alternatively, the sample can be added to MS, DS and dye(s) followed by the addition of CS1 and CS2. CS1 and CS2 need to be added together and either before or after addition of the analyte (e.g., sample). After the sample and HEBA reagents are loaded in the microfluidic device 130, the microfluidic device 130 is incubated for several minutes (e.g., at least 5 minutes and more preferably around 10 minutes or longer). Longer incubation times will produce stronger fluorescent results as the reaction progresses even further. Incubation can take place at or near room temperature. Next as seen in FIG. 10, the microfluidic device 130 is then imaged with an imaging device 140. The imaging device 140 is able to detect fluorescent radiation that is emitted from the wells contained in the microfluidic device 130. The imaging device 140 may include a conventional fluorescent imaging microscope or imager or it may even include a lens-less imager of the type described above.

As seen in FIG. 10, a computing device 150 that is associated with the imaging device 140 (or separate therefrom) can be used to process and analyze the raw images obtained using the imaging device 140. The computing device 150 can identify the positive or “ON” small, fractionated volumes in the microfluidic device 130. A positive fractioned volume may be determined when the intensity signal is±3 s.d. from the mean of unlit fractioned volumes. In the example illustrated in FIG. 10, a portion of the fractionated volumes is illustrated with six volumes identified as being positive. Over the entirety of the fractionated volumes one hundred volumes were identified as being “ON” or positive. In one aspect of the invention, the number of positive or “ON” fractional volumes equates to the number of molecules in the original sample (in this case 100 molecules of analyte in the sample). Of course, depending on the number of fractional volumes there may be a different association between the number of positive fractional volumes and the number of molecules in the sample.

While the analog HEBA assay and digital HEBA assay cover different analyte concentration regimes, in many cases the general range of anticipated concentrations are known in advance such that one can readily choose the particular assay to be used. For example, if one knows that a low concentration of analyte is likely to exist, one can perform the assay using digital HEBA without the need for analog HEBA. Because of the overlap in both the analog and digital HEBA analysis (see FIGS. 9 and 10) a single laboratory instrument or testing device could be used to for both analog HEBA and digital HEBA. For example, the same imaging device (110, 140) could be used along with the same or similar imaging software run by the computing device (120, 150). The particular sample holder device changes depending on whether the assay is analog or digital, but either type of sample holder could be constructed to be imaged by a common imaging device (110, 140). Moreover, as explained herein, the HEBA assay (both analog and digital) encompasses not only detection of an analyte in a sample but it also includes the ability to detect the amount or concentration of the analyte in the sample.

While the HEBA platform has principally been described using reagents or reaction components that are oligonucleotides, it should be appreciated that other molecule types may form various components (e.g., multiplex molecular substrate, catalytic precursors, and/or dummy reactant). Proteins, such as DNA-binding proteins, for example, may form subunits that non-covalently bind to each other, target proteins or target oligonucleotides yet may release target proteins or oligonucleotides to an unbound or free state by binding of a catalytic complex made of two or more catalytic precursors (comprising split oligonucleotides, peptides or small proteins) brought together by an analyte. These unbound target proteins, like target sequences discussed above, can be used, or combined with other reagents such as dyes to generate an optical signal that can be detected. For example a target protein can bind and change the conformation on a reporter protein to move a quencher away from a fluorophore and enable fluorescent emission. Alternatively, a target protein could act as an enzyme when in an unbound state to cleave a separate reporter peptide consisting of a fluorophore and quencher thereby liberating a fluorophore from a nearby quencher. A dummy reactant (protein, peptide or oligonucleotide) is then reacted with the multiplex molecular substrate with bound catalytic complex to displace the catalytic complex back to a free or unbound state suitable for further reactions with the multiplex molecular substrate.

While embodiments of the present invention have been shown and described, various modifications may be made without departing from the scope of the present invention. The invention, therefore, should not be limited, except to the following claims, and their equivalents.

1. A method for detecting an analyte in a sample comprising:
binding a first catalytic precursor to the analyte at a first epitope and binding a second catalytic precursor to the analyte at a second, different epitope to generate a catalytic complex;
reacting the catalytic complex with a multiplex molecular substrate to generate a first target molecule and an intermediate substrate bound to the catalytic complex;
reacting the intermediate substrate with a dummy reactant to generate a second target molecule, wherein the reaction further generates a waste molecule containing the dummy reactant and an unbound catalytic complex; and
detecting an optical signal generated by one or more dye(s) specific to the first target molecule and/or the second target molecule, wherein the overall reaction has substantially net zero enthalpy and a positive entropy change.

2. The method of claim 1, wherein the multiplex molecular substrate comprises a plurality of molecular subunits non-covalently bound to each other.

3. The method of claim 2, wherein the multiplex molecular substrate comprises oligonucleotides.

4. The method of claim 3, wherein the first catalytic precursor comprises an oligonucleotide comprising a first sequence (CS1).

5. The method of claim 3, wherein the second catalytic precursor comprises an oligonucleotide comprising a second sequence (CS2).

6. The method of claim 3, wherein the dummy reactant comprises an oligonucleotide comprising a dummy sequence (DS).

7. The method of claim 3, wherein the waste molecule comprises an oligonucleotide comprising a waste sequence (WS).

8. The method of claim 3, wherein the first target molecule comprises an oligonucleotide comprising a first target sequence (TS1).

9. The method of claim 3, wherein the second target molecule comprises an oligonucleotide comprising a second target sequence (TS2).

10. A method for detecting an analyte in a sample comprising:
binding a first catalytic precursor to the analyte at a first epitope and binding a second catalytic precursor to the analyte at a second, different epitope to generate a catalytic complex;
reacting the catalytic complex with a multiplex molecular substrate to catalytically generate one or more target molecules and a waste molecule, the multiplex molecular substrate comprising a plurality of molecular subunits non-covalently bound to each other; and
detecting an optical signal generated by one or more dye(s) specific to the one or more target molecules, wherein the overall reaction to form the one or more target molecules and the waste molecule has substantially net zero enthalpy and a positive entropy change.

11. The method of claim 10, wherein the first catalytic precursor, the second catalytic precursor, multiplex molecular substrate, the one or more target molecules, and the waste molecule comprise oligonucleotides.

12. A method for detecting an analyte comprising:
binding a first catalytic sequence (CS1) of oligonucleotides to the analyte at a first epitope and binding a second catalytic sequence (CS2) of oligonucleotides to the analyte at a second, different epitope;
reacting the analyte bound with the first catalytic sequence (CS1) and second the catalytic sequence (CS2) with a multiplexed oligonucleotide substrate (MS) to generate a first target oligonucleotide sequence (TS1) and an intermediate substrate containing the first catalytic sequence (CS1) and second the catalytic sequence (CS2);
reacting the intermediate substrate with a single stranded oligonucleotide dummy sequence (DS) to generate a second target oligonucleotide sequence (TS2), wherein the reaction further generates a waste sequence (WS) from the dummy sequence (DS); and
detecting an optical signal generated by dye(s) specific to TS1 and/or TS2.

13. The method of claim 12, wherein the dye comprises an oligonucleotide specific to TS1 or TS2 containing a fluorophore and a quencher.

14. (canceled)

15. The method of claim 12, wherein the analyte binds to the first catalytic sequence (CS1) and the second catalytic sequence (CS2) in one or more of a plurality of droplets containing a sample.

16. The method of claim 12, wherein the analyte binds to the first catalytic sequence (CS1) and the second catalytic sequence (CS2) in one or more of a plurality of wells containing a sample.

17. The method of claim 12, wherein the signal generated by the dye(s) detects the presence of an analyte or the concentration of the analyte in a sample.

18. (canceled)

19. The method of claim 12, wherein the signal generated by a dye specific to TS1 is different from the signal generated by another dye specific to TS2.

20. (canceled)

21. The method of claim 12, wherein the first catalytic sequence (CS1) contains a toehold sequence.

22-25. (canceled)

26. A method of detecting the presence of an analyte in a sample comprising:
generating a plurality of small sample volumes containing a first catalytic sequence (CS1) of oligonucleotides, a second catalytic sequence (CS2) of oligonucleotides, a multiplexed oligonucleotide substrate (MS) containing a first target oligonucleotide sequence (TS1) and a second target oligonucleotide sequence (TS2), a dummy sequence (DS), and one or more dyes specific to TS1 and/or TS2;
incubating the small sample volumes for a period of time; and
detecting an optical signal generated within the small sample volumes.

27. The method of claim 26, wherein the plurality of small sample volumes comprises a plurality of droplets or sample wells.

28. (canceled)

29. The method of claim 26, wherein the dye comprises an oligonucleotide specific to TS1 or TS2 containing a fluorophore and a quencher.

30. (canceled)
